(19) United States
(12) Patent Application Publication
(10) Pub. No.: US 2021/0096075 A1
(43) Pub. Date: Apr. 1, 2021

(54) KIT FOR QUICKLY DETECTING CADMIUM CONTENT IN SAMPLE

(71) Applicant: Beijing Diagreat Biotechnologies Co., ltd., Beijing (CN)

(72) Inventors: Jianping Zhou, Beijing (CN); Yanxin Wang, Beijing (CN); Yujun Zhou, Beijing (CN)

(21) Appl. No.: 16/730,831

(22) Filed: Dec. 30, 2019

(30) Foreign Application Priority Data
Sep. 26, 2019 (CN) 201910916308.3

(51) Int. Cl. G01N 21/64; G01N 33/558; G01N 33/577; G01N 33/68

(52) U.S. Cl. CPC G01N 21/6428; G01N 33/68; G01N 33/577; G01N 33/558

(57) ABSTRACT

The present invention relates to a detection kit for quickly detecting a cadmium content in a sample, and belongs to the technical field of medical in vitro immunoassay. The kit of the present invention includes a detection card and a quality control; the detection card includes a bottom plate, and a sample pad, a glass fiber membrane, a nitrocellulose membrane and an absorbent paper which are arranged on the surface of the bottom plate sequentially from a loading end. The kit of the present invention has advantages of a low detection instrument cost, simple and rapid operations, storage and transportation at room temperature, realization of packaging for a single person, and a good stability.

### TECHNICAL FIELD

The present invention belongs to the technical field of medical in vitro immunoassay, and particularly relates to a detection kit for quickly detecting a cadmium content in a sample.

### BACKGROUND

Cadmium is a heavy metal which is extremely harmful to human body, as discovered by stromcyer in 1817, and is mainly enriched in the body through a food chain. Long-term accumulation of cadmium in the human body will cause renal tubular injury, affect the absorption of phosphorus, calcium and vitamin D, cause osteoporosis, osteomalacia and fracture, change the morphology of the brain central nervous system, interfere with the normal metabolism of iron in the body, induce a cancer, damage the male reproductive system and affect fertility. With the rapid development of industry and agriculture in recent years, the heavy metal pollution in the environment has become increasingly serious. However, the quality and speed requirements for heavy metal detection and analysis are getting higher and higher.

Currently, in 30 provincial and municipal medical institutions of China, in clinical examination a tungsten boat elemental analyzer for lead and cadmium is widely used for measuring blood cadmium. This instrument is an atomic absorption spectrometer specialized for detecting blood cadmium by using the tungsten boat as an atomizer (Beijing Bohui Innovation Biotechnology Co., Ltd.). The measured sample is atomized in the tungsten boat by electric heating, so as to generate a large number of ground-state free atoms, thereby absorbing the characteristic spectral line of a measured element emitted by a hollow cathode lamp, and completing the measurement process. A lens glass cover for an atomization cell will be polluted by the adsorption of smoke. At this time, due to the vibration of a solenoid valve, a contaminant may fall into the tungsten boat and thus cause an error. Therefore, during a large number of continuous detections, the detection should be stopped at any time for cleaning as desired, especially during a large number of detections for detecting samples with a high content of cadmium. As the use number of the tungsten boat increases, the tungsten boat is gradually thinning. It is possible that an error in a detection result is caused due to sample splashing resulting from too high and too fast temperature rising during the work. Therefore, when the tungsten boat has been used for more than 10 times, the temperature of the tungsten boat should be re-adjusted or a new tungsten boat should be replaced, and meanwhile a standard curve is redone; with the use of an element lamp, its luminous intensity will gradually decrease, and its stability may also decrease. Therefore, it is not appropriate to use the element lamp continuously for too many times after completion of the standard curve. A national reference material should be added every 10 servings to monitor the sensitivity of the element lamp. If the sensitivity is reduced or the energy value is unstable, the standard curve should be redone or the element lamp should be replaced. This detection method has disadvantages of being not capable of detecting a sample with a high throughput, requiring frequent replacement of the element lamp, having a high detection cost, and having cumbersome operations.

### SUMMARY

An objective of the present invention is to provide a detection kit for quickly detecting a cadmium content in a sample. The kit of the present invention has advantages of a low detection instrument cost, a rapid detection speed, simple and rapid operations, an available high throughput, storage and transportation at room temperature, realization of packaging for a single person, and a good stability.

The present invention provides a kit for rapidly detecting a cadmium content in a sample, the kit including a detection card and a quality control; where the detection card comprises a bottom plate, and a sample pad, a glass fiber membrane, a nitrocellulose membrane and an absorbent paper which are arranged on the surface of the bottom plate sequentially from a loading end;

the sample pad is treated by soaking in a sample pad treatment buffer, and the sample pad treatment buffer includes an active protein and a surfactant;

the glass cellulose membrane is coated with: a conjugate of a cadmium-specific antibody and a fluorescent microsphere, and a conjugate of a chicken IgY antibody and a fluorescent microsphere; and

the nitrocellulose membrane is marked with a detection line and a quality control line, the detection line is coated with a cadmium-conjugated hapten, and the quality control line is coated with a goat anti-chicken IgY antibody.

Preferably, the sample pad treatment buffer uses one or more of a phosphate buffer, a TRIS hydrochloride buffer, and a glycine buffer as a basal buffer.

Preferably, the active protein includes one or more of bovine serum albumin, casein and ovalbumin.

Preferably, the surfactant includes one or more of Tween 20, lauryl ether Brij35, and Triton X-100.

Preferably, the ratio of the conjugate of the cadmium-specific antibody and the fluorescent microsphere to the conjugate of the chicken IgY antibody and the fluorescent microsphere is (4-6):1.

Preferably, the fluorescent microsphere has a particle size of 100-200 nm, and an Ex excitation wavelength Ex as measured by a fluorescence spectrophotometer is 365 nm; and an emission wavelength Em is 610 nm.

Preferably, the cadmium-specific antibody in the conjugate of the cadmium-specific antibody and the fluorescent microsphere is a cadmium-specific murine monoclonal antibody.

Preferably, a method for preparing the glass cellulose membrane includes: soaking the glass cellulose membrane in a resuspension buffer for 2-3 hours (“h”), and baking it for 1-2 h to obtain a preprocessed glass cellulose membrane; respectively dissolving the conjugate of the cadmium-specific antibody and the fluorescent microsphere and the conjugate of the chicken IgY antibody and the fluorescent microsphere in the resuspension buffer, spraying the solution onto the preprocessed glass cellulose membrane, and baking; wherein the resuspension buffer uses one or more of a carbonate buffer, a TRIS hydrochloride buffer, and a glycine buffer as a basal buffer of 50-200 mM having a pH value of 8.5-9.6, and further includes 0.1-1.0 g/L of sucrose, 0.1-4 g/L of mannitol, and a SDS with a percentage mass content of 0.1%.

Preferably, the quality control is a cadmium-containing buffer, and the buffer includes the following components: a 0.5 mmol/L nitric acid buffer, Tween 20 with a mass-to-volume ratio of 0.1%, bovine serum albumin with a percentage mass content of 0.5%, Proclin300 with a percentage mass content of 0.1%, and water as the solvent, with the pH value being 5.2.

Preferably, the quality control is stored after lyophilization, and a lyophilization buffer for the lyophilization uses a nitrate buffer having a pH value of 5.2-5.4 as a basal buffer, and also comprises 3-8 g/L of sucrose, 5-20 g/L of mannitol and Proclin300 with a percentage mass content of 0.1%.

The present invention provides a detection kit for quickly detecting a cadmium content in a sample. The present invention provides an entirely new immunochromatography method for achieving the detection of the heavy metal cadmium, and has advantages of a low detection instrument cost, simple and rapid operations, storage and transportation at room temperature, realization of packaging for a single person, and a good stability. As compared with a graphite furnace atomic absorption spectrometry which has a large matrix interference and a high price, strict requirements of experimental conditions and demands for a large current, and uses a high-voltage graphite tube that is easily damaged and thus is a consumable, the kit of the present invention applies an immunochromatography which detects rapidly for only 15 min, has a low detection instrument cost, simple operations, and reagent capable of being stored at room temperature; as compared with a biosensor method, the kit of the present invention has the advantages of the low detection instrument cost, simple and rapid operations, reagents capable of being stored at room temperature, packaging for a single person, sample types of serum and urine, good stability and high sensitivity; as compared with an indicator biological method, the kit of the present invention has the advantages of relatively higher specificity and relatively higher sensitivity; as compared with an enzyme assay, the kit of the present invention has advantages of relatively higher specificity and selectivity; as compared with an ELISA immunoassay, the kit of the present invention has advantages of low detection instrument cost, simple and rapid operations, reagents capable of being stored at room temperature, packaging for a single person, sample types of serum and urine, good stability and high sensitivity; and as compared with a method for detecting a cadmium element in whole blood by a tungsten-boat atomic absorption spectrometry (Beijing Bohui Innovation Biotechnology Co., Ltd., granted publication No. CN101592597A), with the principle of the measured sample in the tungsten boat being atomized by electric heating so as to generate a large number of ground-state free atoms, thereby absorbing the characteristic spectral line of a measured element emitted by a hollow cathode lamp, and completing the measurement process, the kit of the present invention has advantages of detecting the content of the cadmium element in serum and urine by the immunochromatography, having a low detection instrument cost, being capable of storing and transporting at room temperature, realizing high-throughput detection, and packing for a single person.

### BRIEF DESCRIPTION OF THE DRAWINGS

FIG. 1 is a calibration curve diagram as provided by Example 1 of the present invention;

FIG. 2 is a diagram showing the correlation of reagents as provided in Comparative Example 1 of the present invention; and

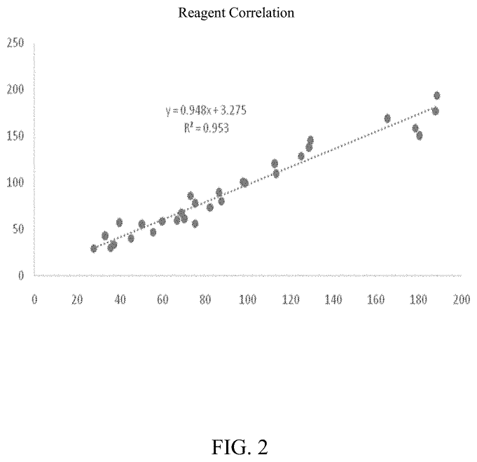

FIG. 3 is a schematic structural view of a detection card in the kit provided by the present invention.

### DESCRIPTION OF THE EMBODIMENTS

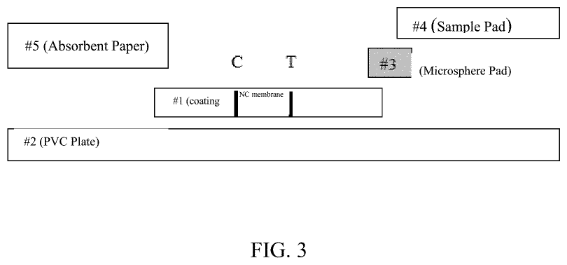

The present invention provides a kit for rapidly detecting a cadmium content in a sample, the kit including a detection card and a quality control; where the detection card includes a bottom plate, and a sample pad, a glass fiber membrane, a nitrocellulose membrane and an absorbent paper which are arranged on the surface of the bottom plate sequentially from a loading end. The detection card of the present invention is as shown in FIG. 3, where 1 represents a nitrocellulose membrane, and specifically a coated NC membrane; 2 represents a bottom plate, and specifically a PVC plate; 3 represents a glass fiber membrane, and specifically a microsphere pad; 4 represents a sample pad; and 5 represents absorbent paper.

In the present invention, the sample pad is treated by soaking in a sample pad treatment buffer, and the sample pad treatment buffer includes an active protein and a surfactant. After treated by soaking in the sample pad treatment buffer, the adsorption of a sample by the sample pad can be reduced. In the present invention, the sample pad treatment buffer uses one or more of a phosphate buffer, a TRIS hydrochloride buffer, and a glycine buffer as a basal buffer. The use of the sample pad treatment buffer in the present invention enables the sample pad to maintain an appropriate ionic strength to cope with a sample having a relatively higher ionic strength, correcting the difference in pH between individual samples. In the present invention, the active protein includes one or more of bovine serum albumin, casein and ovalbumin. The addition of the active protein of the present invention can block an active site on the sample pad, ensuring that target analytes are all flowed out and fully participate in the reaction. In the present invention, the surfactant includes one or more of Tween 20, lauryl ether Brij35, and Triton X-100. The surfactant of the present invention can improve the detection line and sensitivity of metal ion determination and increase the specificity. In the present invention, the time of the soaking treatment is preferably 1-2 h, and more preferably 2 h. In the present invention, after the soaking treatment, the sample pad is preferably subjected to a drying treatment, and preferably baked at 37° C. The present invention has no special limitation on sources of respective components of the sample pad treatment buffer, and a commercially-available product well known to those skilled in the art can be adopted.

In the present invention, the glass cellulose membrane is coated with: a conjugate of a cadmium-specific antibody and a fluorescent microsphere, and a conjugate of a chicken IgY antibody and a fluorescent microsphere. In the present invention, the conjugate of the cadmium-specific antibody and the fluorescent microsphere has the function of specifically recognizing the heavy metal cadmium in the sample, and can form an immune complex with cadmium, and the immune complex is chromatographed along the nitrocellulose membrane into a detection zone (T), and binds with the pre-coated cadmium-conjugated hapten, with its fluorescence intensity being inversely proportional to the Cd content in the sample. The conjugate of the chicken IgY antibody and the fluorescent microsphere is chromatographed into a quality control zone (C) and binds with the pre-coated goat anti-chicken IgY. In the present invention, the volumetric mixture ratio of the conjugate of the cadmium-specific antibody and the fluorescent microsphere to the conjugate of the chicken IgY antibody and the fluorescent microsphere is preferably 4-6:1, and more preferably 5:1. In the present invention, the conjugate of the cadmium-specific antibody and the fluorescent microsphere and the conjugate of the chicken IgY antibody and the fluorescent microsphere are preferably adjusted to have a total mass concentration of 0.2% before being sprayed onto the glass cellulose membrane. In the present invention, the particle size of the fluorescent microsphere is preferably 100-200 nm, and more preferably 100 nm, and the Ex excitation wavelength Ex as measured by a fluorescence spectrophotometer is 365 nm; and the emission wavelength Em is 610 nm. In the present invention, the fluorescent microsphere has advantages of a large difference between exciting and receiving wavelengths, low interference, high detection sensitivity, and good reproducibility. The fluorescent microsphere of the present invention is preferably a conventional commercially-available product. The conjugating method for preparing the conjugate of the cadmium-specific antibody and the fluorescent microsphere and the conjugate of the chicken IgY antibody and the fluorescent microsphere of the present invention is preferably a conventional method. Particularly, in the present invention, the method for preparing the conjugate of the cadmium-specific antibody and the fluorescent microsphere and the conjugate of the chicken IgY antibody and the fluorescent microsphere is as follows: two tubes are taken, the tube 1 is added with 1% of fluorescent microspheres, 10 mg/ml of EDC, 2-10 ug/ml of the cadmium-specific antibody, mixed well for 2 h for conjugation, centrifuged at a speed of 8000-14000 r/min for 30 min, the supernatant is removed, the operations are repeated twice, and then 1% BSA is added for blocking for 1 hour; and the tube 2 is added with 1% of fluorescent microspheres, 10 mg/ml of EDC, and 50 ug of the chicken IgY antibody, mixed well for 2 h for conjugation, centrifuged at a speed of 8000-14000 r/min for 30 min, the supernatant is removed, the operations are repeated twice, and then 1% BSA is added for blocking for 1 hour. In the present invention, the cadmium-specific antibody in the conjugate of the cadmium-specific antibody and the fluorescent microsphere is a cadmium-specific murine monoclonal antibody. In the present invention, a method for preparing the cadmium-specific murine monoclonal antibody preferably includes the following steps: 1. a mouse is injected with an antigen protein (preferably a cadmium-conjugated bovine serum albumin), so that the mouse product is subjected to an immune response; 2. corresponding B lymphocytes are obtained; 3. mouse myeloma cells are fused with the B lymphocytes, and then screened with selective media (HAT and HT); 4. after the screening, the cells are monoclonal cells, which can not only reproduce significantly, but also can produce specific antibodies; 5. the above hybridoma cells are subjected to monoclonal cultivation and antibody detection, such that cells stably secreting monoclonal antibodies can be obtained after multiple times of screening; and 6. the hybridoma cells are cultured at a large scale in vitro, or is intraperitoneally injected into a mouse for propagation to produce ascites, such that a large number of cadmium-specific murine monoclonal antibodies are obtained after purification.

In the present invention, a method for preparing the glass cellulose membrane preferably includes: soaking the glass cellulose membrane in a resuspension buffer for 2-3 h, and baking it for 1-2 h to obtain a preprocessed glass cellulose membrane; respectively dissolving the conjugate of the cadmium-specific antibody and the fluorescent microsphere and the conjugate of the chicken IgY antibody and the fluorescent microsphere in the resuspension buffer, spraying the solution onto the preprocessed glass cellulose membrane, and baking; wherein the resuspension buffer uses one or more of a carbonate buffer, a TRIS hydrochloride buffer, and a glycine buffer as a basal buffer of 50-200 mM having a pH value of 8.5-9.6, and further includes 0.1-1.0 g/L of sucrose, 0.1-4 g/L of mannitol, and a SDS with a percentage mass content of 0.1%. In the present invention, the resuspension buffer is capable of removing unconjugated proteins, and the resuspension buffer provides a suitable pH and an ionic-strength environment to ensure that the final antibody-microsphere conjugate has an activity and the antibody is not easily detached. In the present invention, the baking is preferably baking at 45-65° C. for 2-6 h.

In the present invention, the nitrocellulose membrane is marked with a detection line and a quality control line, the detection line is coated with a cadmium-conjugated hapten, and the quality control line is coated with a goat anti-chicken IgY antibody. In the present invention, the detection line is located on the side closer to the loading end, and the quality control line is located on the side farther from the loading end. In the present invention, the cadmium-conjugated hapten is preferably cadmium-conjugated bovine serum albumin, or cadmium-conjugated ovalbumin. In the present invention, the concentration of the cadmium-conjugated hapten is preferably 1-4 mg/mL, and more preferably 2 mg/mL. The present invention has no special limitation on the source of the cadmium-conjugated hapten, and it may be a conventional commercially-available product, for example purchased from Beijing Deoping Biotechnology Co., Ltd. In the present invention, the concentration of the goat anti-chicken IgY antibody is preferably 0.5-2 mg/mL, and more preferably 1.5 mg/mL. In the present invention, by streaking the cadmium-conjugated hapten and the goat anti-chicken IgY antibody respectively at the position of the detection line and the position of the quality control line on the nitrocellulose membrane, after the membrane streaking is completed, the present invention is preferably dried at 45-65° C. for 2-6 h.

In the present invention, the quality control is a cadmium-containing buffer which can ensure the stability of cadmium nitrate and thus avoid precipitation, and the buffer preferably includes the following components: a 0.5 mmol/L nitric acid buffer, Tween 20 with a mass-to-volume ratio of 0.1%, bovine serum albumin with a percentage mass content of 0.5%, Proclin300 (used as a preservative) with a percentage mass content of 0.1%, and water as the solvent, with the pH value being 5.2. In the present invention, the pH value is selected to be capable of stabilizing the quality control, and suppressing hydrolysis of cadmium nitrate. In the present invention, the quality control is preferably stored after lyophilization, and a lyophilization buffer for the lyophilization uses a nitrate buffer having a pH value of 5.2-5.4 as a basal buffer, and also comprises 3-8 g/L of sucrose, 5-20 g/L of mannitol and Proclin300 with a percentage mass content of 0.1%. The lyophilization buffer of the present invention can ensure the stability of the finished product after lyophilization. In the present invention, the lyophilization is preferably vacuum lyophilization, and the time of the vacuum lyophilization is preferably 12-18 h. The present invention has no specific limitation on the pH adjustment method, and it can be adjusted by using a conventional pH adjuster of which the technical content is well known in the art. In the present invention, preferably a detection card as prepared is used to set values for quality controls, and after the values are assigned, the quality controls are lyophilized separately to obtain quality controls of different concentrations, so as to facilitate the detection of subsequent samples. Particularly, in the present invention, value setting of the quality controls is preferably conducted according to a calibration material which is used for source tracing and value assigning according to a national reference material, and different concentrations of the quality controls are formulated into lyophilization buffers, lyophilized under vacuum for 12-18 hours to obtain the quality controls.

The detection card of the present invention further includes absorbent paper. The present invention has no specific limitation on the absorbent paper, and a conventional commercially-available absorbent paper for a detection card can be adopted.

In the present invention, after the sample pad, the glass fiber membrane, the nitrocellulose membrane, and the absorbent paper are obtained, preferably preparation of the detection card is performed according to a conventional method. For example, the sample pad, the baked glass fiber membrane, the baked nitrocellulose membrane and the absorbent paper are sequentially adhered to the bottom plate; then a bar cutting and casing process is conducted, that is, a large reagent plate is cut and then loaded into a reagent card, added with a desiccant and sealed with an aluminum foil bag, to obtain the detection card.

In the present invention, the detection card preferably further includes a card housing which further includes a back card and an upper cover. The back card is provided with a detection-card slot in which the detection card is embedded. The upper cover is provided with a test window and a sample loading well, where the position of the test window is matched with those of the detection line and the quality control line, and the position of the sample loading well is matched with that of the sample pad. In the present invention, the card housing is preferably a plastic card housing.

In the present invention, the sample that can be detected by the kit includes a blood sample and a urine sample. When the sample is blood, the kit preferably further includes a blood filter membrane. The present invention has no specific limitation on the source of the blood filter membrane, and a conventional commercially-available product of blood filter membrane well known to those skilled in the art can be adopted. The detection method with the kit of the present invention preferably includes the following steps: sampling: 100 ul of a serum sample is taken by a pipette, added into a buffer, mixed well and allowed to standing at room temperature for 5 min, and the supernatant is taken. Loading: a test paper card is removed from a packaging bag, and 80 μL of the dilution of the above sample is taken by a pipette and added into the sample loading well of the test paper card. Testing: After sample loading, the test paper card was allowed to standing for 15 min at room temperature, and then placed into a dry-type fluoroimmunoassay quantitative analyzer to read the data. The time should be strictly controlled within 15 min. The dry-type fluoroimmunoassay quantitative analyzer conducts measuring and analyzing treatments on optical signals to quantify the concentration of the material to be tested.

The kit of the present invention adopts the principle of competitive immunochromatographic detection, and is preferably used in coordinate with the immunoassay quantitative analyzer. The cadmium in a specimen binds with the conjugate of the cadmium-specific antibody and the fluorescent microsphere coated on the glass fiber, to form an immunological complex of “fluorescent particle-antibody-antigen”, which is then chromatographed along the nitrocellulose membrane into the detection zone (T) to bind with the pre-coated cadmium-conjugated hapten on the detection line, with its fluorescence intensity being inversely proportional to the cadmium content in the sample. The conjugate of the chicken IgY antibody and the fluorescent microsphere is chromatographed into the quality control zone (C) and binds with the pre-coated goat anti-chicken IgY antibody on the quality control line. A T/C signal value is calculated via the immunoanalyzer by collecting band fluorescent signals of the detection line (T) and the quality control line (C), and is compared with a standard curve to obtain the content of the heavy metal cadmium in the detected sample. Therefore, as the residual concentration of the heavy metal cadmium in the sample increases, the color development of the detection line is gradually faded as being inhibited; and the goat anti-chicken IgY antibody is recognized and bound since the quality control line contains the goat anti-chicken IgY antibody thereon, and thus a control line will be developed to show that the result of product detecting is valid, no matter whether the sample contains the heavy metal cadmium.

The detection kit for quickly detecting a cadmium content in a sample of the present invention is further described in detail below with reference to specific examples, and the technical solutions of the present invention include, but are not limited to, the following examples.

### Example 1

Preparation of a Reagent Card:

Two tubes were taken, the tube 1 was added with 1% of fluorescent microspheres at 100 nm, 10 mg/ml of EDC, 2.5 ug/ml of the cadmium-specific antibody, mixed well for 2 h for conjugation, centrifuged at a speed of 8000-14000 r/min for 30 min, the supernatant was removed, the operations were repeated twice, and then 1% BSA 2.5 was added for blocking for 1 hour. The tube 2 was added with 1% of fluorescent microspheres, 10 mg/ml of EDC, and 50 ug of the chicken IgY antibody, mixed well for 2 h for conjugation, centrifuged at a speed of 8000-14000 r/min for 30 min, the supernatant was removed, the operations were repeated twice, and then 1% BSA was added for blocking for 1 hour; the prepared labeled conjugates were centrifuged, and re-suspended using 100 mmol of a resuspension buffer containing a TRIS hydrochloride buffer at a pH of 8.0, 0.5 g/L of sucrose, 2 g/L of mannitol, and 0.1% of SDS; the re-suspensions of the tubes 1 and 2 were mixed at 5:1 (volume ratio), and the glass cellulose membrane was immersed in the resuspension buffer for 2 hours, and then baked for 1 hour; and by using a metal spraying membrane-streaking instrument, the label-containing conjugate re-suspension was sprayed onto the baked glass cellulose membrane, and then baked in an air dry oven at 65° C. for 2 hours after the metal spraying was completed;

the nitrocellulose membrane (NC membrane) was cut into 30-31 cm/piece. 2 centrifuge tubes were taken and labeled as C and T, with C being used for the formulation of a coating solution for the goat anti-chicken IgY antibody, and T being used for the formulation of a coating solution for the cadmium-coupled hapten; the centrifuge tube T was added with 0.8 mg/L of the cadmium-coupled hapten (cadmium-BSA coated antigen), diluted by mixing well with a coating buffer (50 mmol of a phosphate buffer with the pH of 8.0) plus a Proclin300 preservative at the volume percentage of 0.1% until the concentration of the cadmium-coupled BSA (purchased from Beijing Deoping Biotechnology Co., Ltd.) was 1 mg/ml; the centrifuge tube C was added with 0.8 mg/L of the goat anti-chicken IgY antibody, then diluted by mixing well with the addition of the coating solution until the concentration of the goat anti-chicken IgY antibody was 1 mg/ml; and the membrane conjugate and the quality control line were streaked on the nitrocellulose membrane respectively at the positions of the T line and the C line, and then baked in an air dry oven at 65° C. for 2 hours after the membrane streaking was completed;

the sample pad, the blood filter membrane, the baked glass cellulose membrane, the baked nitrocellulose membrane, and the absorbent paper were sequentially adhered to the bottom plate; and a large reagent plate was cut and then loaded into a reagent card, added with a desiccant and sealed with an aluminum foil bag, to obtain the test paper card for detection;

Preparation of a Calibration Curve:

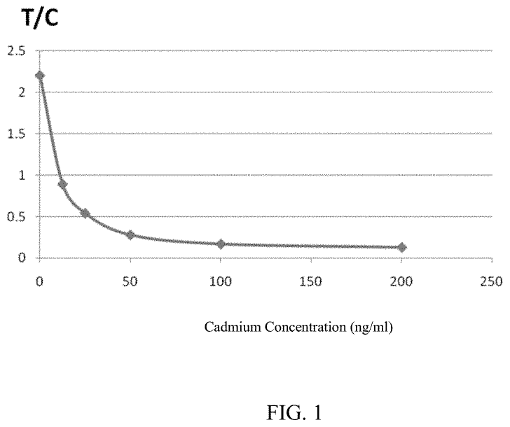

quality controls of concentrations of 0, 12.5, 25, 50, 100, and 200 ng/ml were respectively added dropwise onto the detection card with 3 replicate cards being set for each concentration, mixed well, and allowed to standing and chromatographed for 15 min, and then an immunofluorescence analyzer is used to read fluorescence signal values, so as to calculate the T/C value and thus establish a calibration curve (FIG. 1), where the X axis is the concentration of the quality control and the Y axis is the T/C value.

Detection of Sample Reproducibility:

The detection samples were added dropwise into sample loading wells with 10 replicates were set for each sample. The detection sample was a serum sample. Since a natural high-value sample could not be obtained, the high-value sample was obtained by adding pure cadmium nitrate into a clinical serum sample. Specific detection data of different concentrations of samples were as shown in Table 1:

TABLE 1
Reproducibility of different concentrations of samples

| | Sample Concentration (ug/ml) | | | | | | |
|---|---|---|---|---|---|---|---|
| Sample | Determination | Determination | Determination | Determination | Determination | Determination | Determination |
| Number | 1 | 2 | 3 | 4 | 5 | 6 | 7 |
| 1 | 2 | 2.2 | 1.9 | 1.8 | 2.4 | 2.6 | 1.9 |
| 2 | 5 | 4.9 | 4.8 | 5.2 | 3.9 | 4.8 | 4.6 |
| 3 | 10 | 11.2 | 10.3 | 11.5 | 12.2 | 9.8 | 9.4 |
| 4 | 15 | 16.3 | 15.3 | 13.8 | 15.6 | 16.4 | 17.2 |
| 5 | 23 | 24.7 | 19.9 | 23.4 | 25.6 | 19.9 | 25.8 |
| 6 | 35 | 36.7 | 39.8 | 32.1 | 30.2 | 37.6 | 32.4 |
| 7 | 57 | 55.6 | 57.3 | 57.8 | 58.9 | 51.3 | 50.8 |
| 8 | 68 | 65.4 | 67.2 | 68.3 | 70.1 | 72.1 | 69.8 |
| 9 | 80 | 79.9 | 89.1 | 82.3 | 87.5 | 88.8 | 84.5 |
| 10 | 100 | 99.3 | 98.4 | 89.6 | 101.1 | 98 | 95.4 |

| | Sample Concentration (ug/ml) | | | | | |
|---|---|---|---|---|---|---|
| Sample | Determination | Determination | Determination | Average | Standard | |
| Number | 8 | 9 | 10 | value | Deviation | CV |
| 1 | 1.8 | 2.5 | 1.9 | 2.1 | 0.301846 | 14.37% |
| 2 | 5.3 | 5.7 | 4.8 | 4.9111111 | 0.50111 | 10.20% |
| 3 | 12.1 | 10.3 | 10.5 | 10.73 | 0.968447 | 9.03% |
| 4 | 15.6 | 18.1 | 15.3 | 15.8375 | 0.734725 | 4.64% |
| 5 | 25.9 | 23.4 | 26.2 | 23.78 | 2.347481 | 9.87% |
| 6 | 30.4 | 29.5 | 35.6 | 33.93 | 3.515379 | 10.36% |
| 7 | 49.1 | 53.2 | 56.1 | 54.71 | 3.373903 | 6.17% |
| 8 | 63.2 | 66.9 | 65.1 | 67.61 | 2.647619 | 3.92% |
| 9 | 79.8 | 84.2 | 86.4 | 84.25 | 3.652473 | 4.34% |
| 10 | 89.9 | 94.5 | 99.3 | 96.55 | 4.100203 | 4.25% |

It could be seen from Table 1 that, during the testing of the different concentrations of samples within the whole linear range, the CV of each detection result is less than 15%, indicating a good reproducibility and meeting the testing requirements.

Different concentrations of samples were detected within [2, 200] ng/mL, and the results were as follows:

A sample having a high concentration close to the upper limit of the linear interval and a dilution of the sample were mixed into at least 5 dilution concentrations according to certain ratios with each dilution being tested for 3 times, and an average value of detection results for each dilution was determined separately. The correlation coefficient (r) of linear regression was calculated

TABLE 2
Linearity detected within [2, 200] ng/mL

| Linear Scale | 2 | 10 | 50 | 100 | 200 |
|---|---|---|---|---|---|
| Measured value 1 | 2.2 | 12.1 | 49.8 | 100.2 | 198.3 |
| Measured value 2 | 2.4 | 10.2 | 52.3 | 123.3 | 201.0 |
| Measured value 3 | 1.9 | 13.4 | 48.9 | 118.9 | 212.3 |
| Average value | 2.2 | 11.9 | 50.3 | 114.1 | 203.9 |
| Theoretical Value | 4.09 | 12.32 | 53.45 | 104.86 | 207.68 |
| Relative | −47.04% | −3.39% | −5.83% | 8.84% | −1.84% |
| Deviation | | | | | |
| Absolute | 1.92 | 0.42 | 3.11 | 9.27 | 3.82 |
| Deviation | | | | | |
| Correlation | 0.99796 | | | | |
| Coefficient r | | | | | |

It could be seen from Table 2 that, the correlation coefficient was greater than 0.99, which met the requirements.

A normal sample (C₀) was added with a standard solution formulated from pure cadmium nitrate with a concentration close to a linear high value (200 μg/mL, with the relative deviation being within ±10%). The volume ratio of the added sample C_{S} to the clinical sample C₀ is 1:19, and a sample C was formulated. The samples C₀ and C were measured respectively with 3 times of measurement for each, and then average values were calculated, and the recovery rates were calculated according to equation (1).

$$R = \frac{C \times ( {V}_{0} + V ) - {C}_{0} \times {V}_{0}}{V \times {C}_{S}} \times 100 \% \qquad ( 1 )$$

wherein, R—a recovery rate

V—the volume of the added sample

V₀—the volume of the normal sample

C—the concentration determined after mixing

C₀—the concentration determined for the normal sample

C_{S}—concentration of the added sample

TABLE 3
Data of recovery rate

| | Blood | Mixed | High |
|---|---|---|---|
| 1 | 7.8 | 20.1 | 208.1 |
| 2 | 8.2 | 18.9 | 212.2 |
| 3 | 8.6 | 19.3 | 198.3 |
| Average value | 8.2 | 19.4 | 206.2 |
| Dilution ratio | 19 | 20 | 1 |
| Recovery rate | 113% | | |

As could be seen from Table 3, the recovery rate was between 85-115%, which met the requirements. The accuracy of the reagents as obtained here met the requirements.

### Example 2

Preparation of a Reagent Card:

Two tubes were taken, the tube 1 was added with 1% of fluorescent microspheres at 120 nm, 10 mg/ml of EDC, 1.5 ug/ml of the cadmium-specific antibody, mixed well for 2 h for conjugation, centrifuged at a speed of 8000-14000 r/min for 30 min, the supernatant was removed, the operations were repeated twice, and then 1% BSA 2.5 was added for blocking for 1 hour. The tube 2 was added with 1% of fluorescent microspheres, 10 mg/ml of EDC, and 50 ug of the chicken IgY antibody, mixed well for 2 h for conjugation, centrifuged at a speed of 8000-14000 r/min for 30 min, the supernatant was removed, the operations were repeated twice, and then 1% BSA was added for blocking for 1 hour; the prepared labeled conjugates were centrifuged, and re-suspended using 50 mmol of a resuspension buffer containing a carbonate buffer at a pH of 8.5, 1.0 g/L of sucrose, 4 g/L of mannitol, and 0.1% of SDS; the re-suspensions were mixed at 5:1, and the glass cellulose membrane was immersed in the resuspension buffer for 2 hours, and then baked for 1 hour; and by using a metal spraying membrane-streaking instrument, the label-containing conjugate re-suspension was sprayed onto the baked glass cellulose membrane, and then baked in an air dry oven at 65° C. for 2 hours after the metal spraying was completed;

the T line was the cadmium-coupled BSA at the concentration of 1 mg/ml; the C line was the goat anti-chicken IgY antibody at the concentration of 1 mg/ml; and the membrane conjugate and the quality control line were streaked on the nitrocellulose membrane respectively at the positions of the T line and the C line, and then baked in an air dry oven at 65° C. for 2 hours after the membrane streaking was completed;

the sample pad, the blood filter membrane, the baked golden standard pad, the baked nitrocellulose membrane, and the absorbent paper were sequentially adhered to the bottom plate; and a large reagent plate was cut and then loaded into a reagent card, added with a desiccant and sealed with an aluminum foil bag, to obtain the test paper card for detection;

Preparation of a Calibration Curve:

quality controls of concentrations of 0, 12.5, 25, 50, 100, and 200 ng/ml were respectively added dropwise onto the detection card with 3 replicate cards being set for each concentration, mixed well, and allowed to standing and chromatographed for 15 min, and then an immunofluorescence analyzer is used to read fluorescence signal values, so as to calculate the T/C value and thus establish a calibration curve), where the X axis is the concentration of the quality control and the Y axis is the T/C value.

The calibration curve was prepared as in Example 1.

Reproducibility in duplicate detection of quality controls at two levels:

TABLE 4
Reproducibility of quality controls at two levels

| Quality | Sample Concentration (ng/ml) | | | | | | | | | | Average | Standard | |
|---|---|---|---|---|---|---|---|---|---|---|---|---|---|
| Control | 1 | 2 | 3 | 4 | 5 | 6 | 7 | 8 | 9 | 10 | value | Deviation | CV |
| 1 | 62.5 | 65.9 | 60 | 67 | 56 | 68 | 58 | 62.9 | 60.5 | 73 | 63.28 | 5.188 | 8.2% |
| 2 | 119 | 130 | 126 | 100 | 123 | 131 | 133 | 117 | 125 | 127 | 123.2 | 9.481 | 7.7% |

A sample having a high concentration close to the upper limit of the linear interval and a dilution of the sample were mixed into at least 5 dilution concentrations according to certain ratios with each dilution being tested for 3 times, and an average value of detection results for each dilution was determined separately. The correlation coefficient ® of linear regression was calculated

TABLE 5
Detection of different levels within the linear range

| Linear Scale | 2 | 10 | 50 | 100 | 200 |
|---|---|---|---|---|---|
| Measured value 1 | 1.8 | 11.2 | 51.2 | 98.3 | 187.9 |
| Measured value 2 | 2.5 | 12.8 | 48.9 | 113.2 | 198.3 |
| Measured value 3 | 2.1 | 12.1 | 50.8 | 123.2 | 208.3 |
| Average value | 2.1 | 12.0 | 50.3 | 111.6 | 198.2 |
| Theoretical Value | 4.59 | 12.58 | 52.49 | 102.38 | 202.16 |
| Relative | −53.56% | −4.32% | −4.17% | 8.97% | −1.98% |
| Deviation | | | | | |
| Absolute | 2.46 | 0.54 | 2.19 | 9.19 | 3.99 |
| Deviation | | | | | |
| Correlation | 0.99789 | | | | |
| Coefficient r | | | | | |

The correlation coefficient was greater than 0.99, which met requirements.

### Comparative Example 1

Currently, in 30 provincial and municipal medical institutions of China, in clinical examination a tungsten boat elemental analyzer was widely used. This instrument was an atomic absorption spectrometer specialized for detecting blood cadmium by using the tungsten boat as an atomizer (Beijing Bohui Innovation Biotechnology CO., Ltd.). The measured sample was atomized in the tungsten boat by electric heating, so as to generate a large number of ground-state free atoms, thereby absorbing the characteristic spectral line of a measured element emitted by a hollow cathode lamp, and completing the measurement process. The use time of the tungsten boat should not be too long. Generally, a carbon deposition phenomenon would occur within about 200 times of use, affecting the accuracy of the sample loading. It was not appropriate to use the element lamp continuously for too many times after completion of the standard curve, and a national reference material should be used every 10 servings to monitor the sensitivity of the element lamp.

Correlation of sample results: 76 servings of actual human blood samples were determined for blood cadmium by using the tungsten-boat cadmium element analyzer and the experimental method of the present invention, respectively, and meanwhile a standard material with a concentration value similar to that of the sample to be tested was used as a quality control. The value determined by the tungsten-boat cadmium element analyzer had a range of 28-197 μg/L, and the value determined by the experimental method of the present invention had a range of 27.9-188 μg/L.

The following was a comparison of data for blood cadmium as determined by two methods

TABLE 6
Comparison with cadmium data determined by
the tungsten-boat cadmium element analyzer

| Sample | Test | Control |
|---|---|---|
| Number | Reagent | Reagent |
| 1 | 27.9 | 29.3 |
| 2 | 35.7 | 30.1 |
| 3 | 37.3 | 33.3 |
| 4 | 45.3 | 39.9 |
| 5 | 33.2 | 42.8 |
| 6 | 55.7 | 46.4 |
| 7 | 50.3 | 55.3 |
| 8 | 39.8 | 57.1 |
| 9 | 66.8 | 59.5 |
| 10 | 59.9 | 58.3 |
| 11 | 75.3 | 55.9 |
| 12 | 70.3 | 61.2 |
| 13 | 68.9 | 67.3 |
| 14 | 82.3 | 73.3 |
| 15 | 75.3 | 77.8 |
| 16 | 87.6 | 79.8 |
| 17 | 73.2 | 85.9 |
| 18 | 86.5 | 89.6 |
| 19 | 98.6 | 99.7 |
| 20 | 97.7 | 100.9 |
| 21 | 113.2 | 109.7 |
| 22 | 112.6 | 120.3 |
| 23 | 125 | 128.3 |
| 24 | 128.7 | 137.8 |
| 25 | 129.3 | 145.3 |
| 26 | 180.3 | 150.3 |
| 27 | 178.5 | 158.6 |
| 28 | 165.3 | 168.9 |
| 29 | 187.9 | 176.9 |
| 30 | 188.5 | 193.2 |

The linear correlation was greater than 0.9, which met clinical requirements.

It was as shown in Table 6. The correlation coefficient of cadmium data determined by the experimental method of the present invention and the tungsten-boat cadmium element analyzer was greater than 0.9, and a rapid speed, a high throughput, a single-person packaging, good stability of the reagent when stored at room temperature, and high reproducibility could be realized.

The above description is only a preferred embodiment of the present invention, and it should be noted that those skilled in the art can also make several improvements and modifications without departing from the principles of the present invention. It should be considered as the scope of protection of the present invention.

What is claimed is:

1. A kit for rapidly detecting a cadmium content in a sample, the kit comprising a detection card and a quality control; wherein the detection card comprises a bottom plate, and a sample pad, a glass fiber membrane, a nitrocellulose membrane and an absorbent paper which are arranged on the surface of the bottom plate sequentially from a loading end;
the sample pad is treated by soaking in a sample pad treatment buffer, and the sample pad treatment buffer comprises an active protein and a surfactant;
the glass cellulose membrane is coated with: a conjugate of a cadmium-specific antibody and a fluorescent microsphere, and a conjugate of a chicken IgY antibody and a fluorescent microsphere; and
the nitrocellulose membrane is marked with a detection line and a quality control line, the detection line is coated with a cadmium-conjugated hapten, and the quality control line is coated with a goat anti-chicken IgY antibody.

2. The kit according to claim 1, wherein the sample pad treatment buffer uses one or more of a phosphate buffer, a TRIS hydrochloride buffer, and a glycine buffer as a basal buffer.

3. The kit according to claim 1, wherein the active protein comprises one or more of bovine serum albumin, casein and ovalbumin.

4. The kit according to claim 1, wherein the surfactant comprises one or more of Tween 20, lauryl ether Brij35, and Triton X-100.

5. The kit according to claim 1, wherein the ratio of the conjugate of the cadmium-specific antibody and the fluorescent microsphere to the conjugate of the chicken IgY antibody and the fluorescent microsphere is (4-6):1.

6. The kit according to claim 1, wherein the fluorescent microsphere has a particle size of 100-200 nm, and an Ex excitation wavelength Ex as measured by a fluorescence spectrophotometer is 365 nm; and an emission wavelength Em is 610 nm.

7. The kit according to claim 5, wherein the fluorescent microsphere has a particle size of 100-200 nm, and an Ex excitation wavelength Ex as measured by a fluorescence spectrophotometer is 365 nm; and an emission wavelength Em is 610 nm.

8. The kit according to claim 1, wherein the cadmium-specific antibody in the conjugate of the cadmium-specific antibody and the fluorescent microsphere is a cadmium-specific murine monoclonal antibody.

9. The kit according to claim 5, wherein the cadmium-specific antibody in the conjugate of the cadmium-specific antibody and the fluorescent microsphere is a cadmium-specific murine monoclonal antibody.

10. The kit according to claim 1, wherein a method for preparing the glass cellulose membrane comprises: soaking the glass cellulose membrane in a resuspension buffer for 2-3 hours (h), and baking it for 1-2 h to obtain a preprocessed glass cellulose membrane; respectively dissolving the conjugate of the cadmium-specific antibody and the fluorescent microsphere and the conjugate of the chicken IgY antibody and the fluorescent microsphere in the resuspension buffer, spraying the solution onto the preprocessed glass cellulose membrane, and baking; wherein the resuspension buffer uses one or more of a carbonate buffer, a TRIS hydrochloride buffer, and a glycine buffer as a basal buffer of 50-200 mM having a pH value of 8.5-9.6, and further comprises 0.1-1.0 g/L of sucrose, 0.1-4 g/L of mannitol, and a SDS with a percentage mass content of 0.1%.

11. The kit according to claim 1, wherein the quality control is a cadmium-containing buffer, and the buffer comprises the following components: a 0.5 mmol/L nitric acid buffer, Tween 20 with a mass-to-volume ratio of 0.1%, bovine serum albumin with a percentage mass content of 0.5%, Proclin300 with a percentage mass content of 0.1%, and water as the solvent, with the pH value being 5.2.

12. The kit according to claim 1, wherein the quality control is stored after lyophilization, and a lyophilization buffer for the lyophilization uses a nitrate buffer having a pH value of 5.2-5.4 as a basal buffer, and also comprises 3-8 g/L of sucrose, 5-20 g/L of mannitol and Proclin300 with a percentage mass content of 0.1%.

13. The kit according to claim 9, wherein the quality control is stored after lyophilization, and a lyophilization buffer for the lyophilization uses a nitrate buffer having a pH value of 5.2-5.4 as a basal buffer, and also comprises 3-8 g/L of sucrose, 5-20 g/L of mannitol and Proclin300 with a percentage mass content of 0.1%.
